(19) United States
(12) Patent Application Publication
(10) Pub. No.: US 2025/0064512 A1
(43) Pub. Date: Feb. 27, 2025

(54) PERICARDIAL TRANSECTION DEVICE WITH SHAPE SET ELECTRODE

(71) Applicant: EDWARDS LIFESCIENCES CORPORATION, Irvine, CA (US)

(72) Inventors: Yuval Kasher, Kfar Shmuel (IL); Gideon Meyer-Brodnitz, Ramot Menashe (IL)

(21) Appl. No.: 18/945,385

(22) Filed: Nov. 12, 2024

Related U.S. Application Data
(63) Continuation of application No. PCT/US2023/022970, filed on May 19, 2023.
(60) Provisional application No. 63/346,510, filed on May 27, 2022.

(51) Int. Cl. A61B 18/14; A61B 17/00; A61B 17/32; A61B 18/00

(52) U.S. Cl. CPC A61B 18/1492; A61B 17/32002; A61B 2017/00243; A61B 2018/00363; A61B 2018/00595; A61B 2018/00601

(57) ABSTRACT

A medical device includes a flexible catheter including a distal end, at least one lumen, and a longitudinal axis, an incision device coupled to the distal end of the catheter, and a distal tip coupled to and projecting from the incision assembly. The incision device comprises a wire that includes one or more electrodes for creating an incision through a pericardium. The wire has a first end fixed to an external surface of the distal tip and a second end extending through the distal opening of the distal tip. The wire is structured such that in a deployed configuration, (i) the portion of the wire extending from the first end of the wire to the distal opening of the body of the distal tip forms a shape comprising a protrusion extending relative to a longitudinal axis of the transcatheter and (ii) the electrode is positioned proximal to the protrusion.

### RELATED APPLICATIONS

This application is a continuation application of International Patent Application No. PCT/US2023/022970, filed May 19, 2023, which claims the benefit of U.S. Provisional Application No. 63/346,510, filed on May 27, 2022. Each of the foregoing patent applications are hereby incorporated by reference in their entireties.

### TECHNICAL FIELD

This disclosure is directed to devices and methods for cutting in the pericardium. The devices and methods are generally applicable to the treatment of heart failure, for example, heart failure with preserved ejection fraction (HFpEF) or reduced injection fraction (HFrEF) by introducing one or more incision lengths in a pericardium, e.g., a parietal layer.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In one aspect, the present invention embraces a medical device for creating incisions within a body tissue, such as a pericardium, including but not limited to incisions in the parietal layer. In some example embodiments, alone or in combination with any previous embodiment, the medical device includes a transcatheter including at least one lumen, a longitudinal axis, a proximal end, and a distal end, a distal tip adjacent the distal end of the transcatheter, where the distal tip includes a body having an external surface and a distal opening, an incision device operably coupled to the distal end of the transcatheter, and a biasing member operably coupled to the second end of the wire. The incision device includes a wire including a first end fixed to the external surface of the body of the distal tip, a second end extending through the distal opening of the body of the distal tip, and an electrode. The wire has a first configuration in which a portion of the wire extending from the first end to the distal opening of the body of the distal tip is in contact with or abuts the external surface of the body and a second configuration in which (i) the portion of the wire extending from the first end to the distal opening of the body of the distal tip forms a shape including a protrusion extending at an angle relative to the longitudinal axis of the transcatheter (in some instances extending substantially perpendicular to the longitudinal axis) and (ii) the electrode is positioned proximal to the protrusion. In some example embodiments, alone or in combination with any previous embodiment, the biasing member is structured to advance at least a portion of the wire through the distal opening of the body of the distal tip to move the wire from the first configuration to the second configuration.

In some example embodiments, alone or in combination with any previous embodiment, the wire is shape-set to form the second configuration.

In some example embodiments, alone or in combination with any previous embodiment, the biasing member is structured to retract at least a portion of the wire through the distal opening of the body of the distal tip to move the wire from the second configuration to the first configuration.

In some example embodiments, alone or in combination with any previous embodiment, the electrode is structured to receive current and/or radio frequency energy to ablate, burn, vaporize, and/or separate tissue.

In some example embodiments, alone or in combination with any previous embodiment, after an incision is formed within the pericardium, when the wire is in the second configuration, and when the incision device is positioned beneath the pericardium, the protrusion extends through the incision.

In some example embodiments, alone or in combination with any previous embodiment, when the wire is in the second configuration, the electrode is positioned on the wire adjacent a base of the protrusion.

In some example embodiments, alone or in combination with any previous embodiment, the wire includes a drawn filled tube (DFT).

In some example embodiments, alone or in combination with any previous embodiment, the wire includes a core within a shell, where the shell is less conductive than the core.

In some example embodiments, alone or in combination with any previous embodiment, the wire includes a core within a shell, where the shell is nitinol and the core is silver.

In some example embodiments, alone or in combination with any previous embodiment, the wire includes a core within a shell, where the electrode is formed by removing a portion of the shell.

In some example embodiments, alone or in combination with any previous embodiment, the wire includes a frame, where the electrode and a wire are attached to the frame.

In some example embodiments, alone or in combination with any previous embodiment, the wire includes a frame having a rectangular cross-section.

In some example embodiments, alone or in combination with any previous embodiment, the wire includes a nitinol frame.

In some example embodiments, alone or in combination with any previous embodiment, the transcatheter is steerable.

In some example embodiments, alone or in combination with any previous embodiment, at least a portion of the transcatheter is radiopaque.

In some example embodiments, alone or in combination with any previous embodiment, the at least one lumen includes a guidewire lumen extending through the incision device.

In some example embodiments, alone or in combination with any previous embodiment, at least a portion of the incision device is radiopaque.

In some example embodiments, alone or in combination with any previous embodiment, the incision device includes a rigid housing and a cutting surface received by the rigid housing.

In some example embodiments, alone or in combination with any previous embodiment, the rigid housing is metal.

In some example embodiments, alone or in combination with any previous embodiment, the biasing member is positioned in the incision device.

In some example embodiments, alone or in combination with any previous embodiment, the biasing member exerts a rotary force or torque to the cutting surface.

In some example embodiments, alone or in combination with any previous embodiment, the medical device includes an actuator coupled to the biasing member.

In some example embodiments, alone or in combination with any previous embodiment, the medical device includes a guidewire slidably positioned within the guidewire lumen.

In some example embodiments, alone or in combination with any previous embodiment, the distal tip includes a lumen operably coupled to the guidewire lumen.

In some example embodiments, alone or in combination with any previous embodiment, a distal tip lumen slidably receives the guidewire.

In some example embodiments, alone or in combination with any previous embodiment, at least a portion of the distal tip is radiopaque.

In some example embodiments, alone or in combination with any previous embodiment, the medical device includes a sheath, where the sheath has a distal end being slidably locatable on the transcatheter.

In some example embodiments, alone or in combination with any previous embodiment, at least a portion of the distal end of the sheath is radiopaque.

In some example embodiments, alone or in combination with any previous embodiment, the sheath includes at least one opening adjacent the distal end of the sheath.

In some example embodiments, alone or in combination with any previous embodiment, the distal end of the sheath is traversable along the transcatheter to align with the at least one opening of the transcatheter.

In some example embodiments, alone or in combination with any previous embodiment, the at least one opening of the sheath is traversable along the transcatheter to cover the at least one opening of the transcatheter or to uncover the at least one opening of the transcatheter.

In some example embodiments, alone or in combination with any previous embodiment, at least a portion of the distal end of the sheath and at least a portion of the at least one opening of the transcatheter is radiopaque to align the distal end of the sheath and the at least one opening of the transcatheter.

In some example embodiments, alone or in combination with any previous embodiment, at least a portion of a circumference of the at least one opening of the sheath and at least a portion of a circumference of the at least one opening of the transcatheter is radiopaque to align their respective openings.

In some example embodiments, alone or in combination with any previous embodiment, the at least one opening of the sheath is traversable to align with the at least one opening of the transcatheter, allowing the cutting surface to laterally project the through both the sheath and the transcatheter.

In some example embodiments, alone or in combination with any previous embodiment, the at least one opening of the sheath is traversable to align with the at least one opening of the transcatheter, allowing the actuator to cause the cutting surface to laterally project the through both the sheath and the transcatheter.

In some example embodiments, alone or in combination with any previous embodiment, the medical device includes one or more stabilizing members located adjacent the at least one opening of the transcatheter.

In some example embodiments, alone or in combination with any previous embodiment, one or more stabilizing members reversibly projects laterally about 120 degrees radially apart about the transcatheter.

In some example embodiments, alone or in combination with any previous embodiment, one or more stabilizing members is a wire, loop, or shape-memory metal.

In some example embodiments, alone or in combination with any previous embodiment, one or more stabilizing members is one or more inflatable structures.

In some example embodiments, alone or in combination with any previous embodiment, the distal end of the sheath is traversable along the transcatheter to uncover the at least one opening of the transcatheter allowing the one or more stabilizing members to laterally project through the one or more openings of the transcatheter.

In some example embodiments, alone or in combination with any previous embodiment, the distal end of the sheath concurrently or sequentially allows the cutting surface and the one or more stabilizing members to laterally project through the one or more openings of the transcatheter.

In some example embodiments, alone or in combination with any previous embodiment, the one or more stabilizing members is operably coupled to the actuator.

In some example embodiments, alone or in combination with any previous embodiment, the one or more openings of the sheath is traversable along the transcatheter allowing the actuator to cause the one or more stabilizing members to laterally project through both the transcatheter and the sheath.

In some example embodiments, alone or in combination with any previous embodiment, the actuator concurrently or sequentially laterally projects the incision device and the one or more stabilizing members through the one or more openings of the transcatheter and the one or more openings of the sheath.

In some example embodiments, alone or in combination with any previous embodiment, at least a portion of the incision device includes an electrode.

In some example embodiments, alone or in combination with any previous embodiment, at least a portion of a cutting surface includes an electrode.

In some example embodiments, alone or in combination with any previous embodiment, the electrode is a wire.

In some example embodiments, alone or in combination with any previous embodiment, the wire is shaped as one or more arcs projecting laterally through the transcatheter along the longitudinal axis.

In some example embodiments, alone or in combination with any previous embodiment, the cutting surface includes a scalpel.

In some example embodiments, alone or in combination with any previous embodiment, at least a portion of the scalpel includes an electrode.

In some example embodiments, alone or in combination with any previous embodiment, the at least one electrode is electrically couplable to a source of radiofrequency energy or electrical current sufficient to cut, separate, scissor, or evaporate a portion of the parietal layer.

In some example embodiments, alone or in combination with any previous embodiment, the medical device includes a second electrode adjacent to the incision device.

In some example embodiments, alone or in combination with any previous embodiment, the second electrode is operably coupled to a source of radiofrequency energy or electrical current.

In some example embodiments, alone or in combination with any previous embodiment, a cutting surface when laterally projected, faces away from the distal end and towards the proximal end of the transcatheter.

In some example embodiments, alone or in combination with any previous embodiment, at least a portion of cutting surface is reversibly adjustable laterally relative to the longitudinal axis of the transcatheter between a range of angles.

In some example embodiments, alone or in combination with any previous embodiment, the range of angles is reversibly adjustable for providing a scissor cut of parietal layer tissue.

In some example embodiments, alone or in combination with any previous embodiment, the medical device includes at least one nerve detection device.

In some example embodiments, alone or in combination with any previous embodiment, at least one nerve detection device is located on the transcatheter.

In some example embodiments, alone or in combination with any previous embodiment, at least one nerve detection device is located adjacent the incision device.

In some example embodiments, alone or in combination with any previous embodiment, at least one nerve detection device is located on the distal tip.

In some example embodiments, alone or in combination with any previous embodiment, at least one nerve detection device is located on a cutting surface.

In some example embodiments, alone or in combination with any previous embodiment, the medical device includes at least one nerve stimulation device. In some example embodiments, alone or in combination with any previous

embodiment, at least one nerve detection device is located on the transcatheter.

In some example embodiments, alone or in combination with any previous embodiment, at least one nerve detection device is located adjacent the incision device.

In some example embodiments, alone or in combination with any previous embodiment, at least one nerve detection device is located on the distal tip.

In some example embodiments, alone or in combination with any previous embodiment, at least one nerve detection device is located on the cutting surface.

In some example embodiments, alone or in combination with any previous embodiment, the medical device includes at least one nerve stimulation device.

In some example embodiments, alone or in combination with any previous embodiment, at least one nerve stimulation device is located on the transcatheter.

In some example embodiments, alone or in combination with any previous embodiment, at least one nerve stimulation device is located adjacent the incision device.

In some example embodiments, alone or in combination with any previous embodiment, at least one nerve stimulation device is located on the distal tip.

In some example embodiments, alone or in combination with any previous embodiment, at least one nerve stimulation device is located on the cutting surface.

In some example embodiments, alone or in combination with any previous embodiment, a kit includes the medical device, a sheath, a guidewire, and a distal tip.

In another aspect, the present invention embraces a method of improving a heart function in a heart of a subject having heart dysfunction. The method includes creating at least one incision length through a pericardium and reducing pressure exerted by the pericardium on a heart.

In some example embodiments, alone or in combination with any previous embodiment, the creation of the at least one incision length through the pericardium causes a reduction in pressure exerted on a heart by the pericardium.

In some example embodiments, alone or in combination with any previous embodiment, the incision length is created in at least one of a parietal layer or fibrous layer of the pericardium.

In some example embodiments, alone or in combination with any previous embodiment, the incision length is created in adipose tissue or fat deposits.

In some example embodiments, alone or in combination with any previous embodiment, the at least one incision length is along a length or circumference of only the parietal layer of the pericardium.

In some example embodiments, alone or in combination with any previous embodiment, the at least one incision length is made in the parietal layer from an anterior to a posterior of a heart.

In some example embodiments, alone or in combination with any previous embodiment, the at least one incision length is made in the parietal layer from a posterior base to an apex of a heart.

In some example embodiments, alone or in combination with any previous embodiment, the at least one incision length is made in the parietal layer from a posterior right atrium to an apex of a heart.

In some example embodiments, alone or in combination with any previous embodiment, the at least one incision length is made in the parietal layer from a left ascending aorta to an apex of a heart.

In some example embodiments, alone or in combination with any previous embodiment, the at least one incision length is made in the parietal layer from a right ascending aorta to an apex of a heart.

In some example embodiments, alone or in combination with any previous embodiment, the at least one incision length is made in the parietal layer transversely about a heart.

In some example embodiments, alone or in combination with any previous embodiment, the method includes, before creating the incision length, puncturing pericardial tissue and providing an access point into a pericardial space.

In some example embodiments, alone or in combination with any previous embodiment, the method includes, before puncturing, providing subxiphoid access to the pericardium.

In some example embodiments, alone or in combination with any previous embodiment, the method includes, before puncturing, providing transvascular access to the pericardium.

In some example embodiments, alone or in combination with any previous embodiment, the method includes, after puncturing, inserting a guidewire into the pericardial space.

In some example embodiments, alone or in combination with any previous embodiment, the method includes advancing a dilator over the guidewire and into the pericardial space.

In some example embodiments, alone or in combination with any previous embodiment, the method includes, after inserting a guidewire or dilator into the pericardial tissue, advancing a transcatheter device over the guidewire into the pericardial space, the transcatheter device having a proximal end, a distal end, and a longitudinal axis.

In some example embodiments, alone or in combination with any previous embodiment, the transcatheter device includes an incision device.

In some example embodiments, alone or in combination with any previous embodiment, the transcatheter device includes a pericardial incision assembly.

In some example embodiments, alone or in combination with any previous embodiment, the pericardial incision assembly includes the incision device.

In some example embodiments, alone or in combination with any previous embodiment, the method includes sterilizing the incision device.

In some example embodiments, alone or in combination with any previous embodiment, the incision device is sterilized via E-beam sterilization.

In some example embodiments, alone or in combination with any previous embodiment, the incision device is sterilized via gamma sterilization.

In some example embodiments, alone or in combination with any previous embodiment, the incision device is sterilized via ethylene oxide sterilization.

In some example embodiments, alone or in combination with any previous embodiment, the incision device is sterilized via autoclave sterilization.

In some example embodiments, alone or in combination with any previous embodiment, the incision device is selected from a group consisting of a scalpel, a mechanical cutting device, an electrosurgical device, a reversibly retractable knife blade and combinations thereof.

In some example embodiments, alone or in combination with any previous embodiment, the method includes obtaining visual information.

In some example embodiments, alone or in combination with any previous embodiment, the method includes advancing a sheath over the transcatheter device, and into the pericardial space.

In some example embodiments, alone or in combination with any previous embodiment, the method includes, after advancing the transcatheter device into the pericardial space, creating an opening at least through a parietal layer from within the pericardial space.

In some example embodiments, alone or in combination with any previous embodiment, creating an opening through the parietal layer is performed using the scalpel, the mechanical cutting device, the electrosurgical device, the reversibly retractable knife blade, or a combination thereof.

In some example embodiments, alone or in combination with any previous embodiment, the method includes, after creating the opening in the parietal layer, introducing the scalpel, the mechanical cutting device, the electrosurgical device, or the reversibly retractable knife blade into the opening, where the scalpel, the mechanical cutting device, the electrosurgical device, or the reversibly retractable knife blade and reverse cutting the pericardium.

In some examples, the method includes, after advancing the transcatheter device into the pericardial space, stabilizing a portion of the transcatheter device within the pericardial space.

In some example embodiments, alone or in combination with any previous embodiment, the stabilizing includes deploying one or more stabilizing members, the one or more stabilizing members projecting laterally from the transcatheter device.

In some example embodiments, alone or in combination with any previous embodiment, one or more stabilizing members is adjacent the distal end of transcatheter device.

In some example embodiments, alone or in combination with any previous embodiment, one or more stabilizing members is selected from a wire, loop, or shape-memory metal.

In some example embodiments, alone or in combination with any previous embodiment, one or more stabilizing members is one or more inflatable structures.

In some example embodiments, alone or in combination with any previous embodiment, the method includes, after stabilizing the transcatheter device into the pericardial space, securing a portion of the transcatheter device within the pericardial space.

In some example embodiments, alone or in combination with any previous embodiment, the method includes, after stabilizing the transcatheter device in the pericardial space, creating an opening through the parietal layer from within the pericardial space using the scalpel, the mechanical cutting device, the electrosurgical device, or the reversibly retractable knife blade.

In some example embodiments, alone or in combination with any previous embodiment, the method includes, after stabilizing the transcatheter device, securing a portion of the transcatheter device in the pericardial space prior to or during either or both of creating an opening through the parietal layer and creating the incision length.

In some example embodiments, alone or in combination with any previous embodiment, the method includes introducing the scalpel, the mechanical cutting device, the electrosurgical device, or the reversibly retractable knife blade into the opening, the scalpel, the mechanical cutting device, the electrosurgical device, or the reversibly retractable knife blade arranged for reverse cutting.

In some example embodiments, alone or in combination with any previous embodiment, the electrosurgical device includes a cutting surface with one or more electrodes.

In some example embodiments, alone or in combination with any previous embodiment, the method includes, after introducing the scalpel, the mechanical cutting device, the electrosurgical device, or the reversibly retractable knife blade to the opening, creating the at least one incision length by cutting, perforating, scissoring, and/or shearing.

In some example embodiments, alone or in combination with any previous embodiment, the at least one incision length begins at the opening and end at the access point.

In some example embodiments, alone or in combination with any previous embodiment, creating the at least one incision length includes creating a plurality of incision lengths.

In some example embodiments, alone or in combination with any previous embodiment, a plurality of incision lengths is isolated from each other.

In some example embodiments, alone or in combination with any previous embodiment, at least one of a plurality of incision lengths intersects each other.

In some example embodiments, alone or in combination with any previous embodiment, the method includes, after creating the at least one incision length, cauterizing at least a portion of the incision length.

In some example embodiments, alone or in combination with any previous embodiment, the method includes, after advancing the transcatheter device into the pericardial space, probing to ascertain a location of a nerve.

In some example embodiments, alone or in combination with any previous embodiment, probing ascertains whether at least a portion of a phrenic nerve is proximate an incision device.

In some example embodiments, alone or in combination with any previous embodiment, probing includes a nerve stimulating device.

In some example embodiments, alone or in combination with any previous embodiment, probing includes a nerve detecting device.

In some example embodiments, alone or in combination with any previous embodiment, creating the at least one incision length is determined in response to a signal indicative of a reduction of restraint of the heart.

In some example embodiments, alone or in combination with any previous embodiment, creating the at least one incision length is determined in response to a signal indicative of a reduction of restraint of the heart, and the method includes repeating the creating of the at least one incision length.

In some example embodiments, alone or in combination with any previous embodiment, the method includes, after creating the at least one incision length, confirming a location of a distal end of the transcatheter device and, in response to a signal indicative of a reduction of restraint of the heart, repeating the steps of creating the at least one incision length, and confirming a location of the distal end.

In yet another aspect, the present invention embraces a medical device including a flexible catheter and an incision device. In some example embodiments, alone or in combination with any previous embodiment, the flexible catheter includes a distal end, at least one lumen, and a longitudinal axis. Additionally, or alternatively, the incision device is operably coupled to the distal end of the transcatheter. In some example embodiments, alone or in combination with any previous embodiment, the incision device includes a base portion including a distal end, where the base portion extends in a distal direction substantially parallel to the longitudinal axis. Additionally, or alternatively, the incision device includes an upper portion extending in a proximal direction substantially parallel to the longitudinal axis. In some example embodiments, alone or in combination with any previous embodiment, the incision device includes a connecting portion joining the upper portion to the distal end of the base portion such that the base portion, the upper portion, and the connecting portion form a confinement space. Additionally, or alternatively, the incision device includes an electrode oriented toward the confinement space.

In some example embodiments, alone or in combination with any previous embodiment, the base portion, the upper portion, and the connecting portion is formed from one or more wires, and the connecting portion includes a bend in the one or more wires.

In some example embodiments, alone or in combination with any previous embodiment, the base portion, the upper portion, and the connecting portion is formed from one or more wires, and a wire, of the one or more wires, adjacent the confinement space includes a core within a shell, where the electrode is formed by removing a portion of the shell.

In some example embodiments, alone or in combination with any previous embodiment, the connecting portion includes a sliding joint permitting a distance between the upper portion and the base portion to increase and decrease.

In some example embodiments, alone or in combination with any previous embodiment, the electrode is structured to receive current and/or radio frequency energy to ablate, burn, vaporize, and/or separate tissue.

In some example embodiments, alone or in combination with any previous embodiment, the electrode is a first electrode, and the medical device includes a second electrode.

In some example embodiments, alone or in combination with any previous embodiment, the electrode is a first electrode positioned on the base portion, and the medical device includes a second electrode, where the second electrode is positioned opposite the first electrode and oriented toward the confinement space.

In some example embodiments, alone or in combination with any previous embodiment, the electrode is a positive electrode, and the medical device includes a negative electrode oriented toward the confinement space.

In some example embodiments, alone or in combination with any previous embodiment, the electrode is a positive electrode, and the medical device includes a negative electrode oriented toward the confinement space, where the positive electrode and the negative electrode are oriented in a same direction toward the confinement space.

In some example embodiments, alone or in combination with any previous embodiment, the flexible catheter is steerable.

In some example embodiments, alone or in combination with any previous embodiment, at least a portion of the flexible catheter is radiopaque.

In some example embodiments, alone or in combination with any previous embodiment, at least one lumen includes a guidewire lumen.

In some example embodiments, alone or in combination with any previous embodiment, an incision assembly includes a housing, an opening in the housing, and a cutting surface.

In some example embodiments, alone or in combination with any previous embodiment, at least a portion of an incision assembly is radiopaque.

In some example embodiments, alone or in combination with any previous embodiment, at least a portion of a circumference of the opening of the housing of the incision assembly is radiopaque.

In some example embodiments, alone or in combination with any previous embodiment, an incision assembly includes a lumen operably coupled to the guidewire lumen.

In some example embodiments, alone or in combination with any previous embodiment, a distal tip includes a lumen operably coupled to the guidewire lumen.

In some example embodiments, alone or in combination with any previous embodiment, the flexible catheter, incision assembly, and distal tip slidably receives a guidewire.

In some example embodiments, alone or in combination with any previous embodiment, the incision assembly is structured for introduction over the guidewire.

In some example embodiments, alone or in combination with any previous embodiment, the incision assembly has a first configuration with the cutting surface received in the incision assembly, and a second configuration where the cutting surface is projected laterally outward from the incision assembly.

In some example embodiments, alone or in combination with any previous embodiment, the incision assembly is operably coupled to a controller, the controller positioned adjacent a proximal end of the flexible catheter.

In some example embodiments, alone or in combination with any previous embodiment, the incision assembly includes a biasing member operably coupled to the cutting surface.

In some example embodiments, alone or in combination with any previous embodiment, the biasing member is operably coupled to the controller.

In some example embodiments, alone or in combination with any previous embodiment, the biasing member exerts a force or torque to the cutting surface.

In some example embodiments, alone or in combination with any previous embodiment, a force or torque is rotary.

In some example embodiments, alone or in combination with any previous embodiment, the biasing member is a spring or band.

In some example embodiments, alone or in combination with any previous embodiment, a spring is a torsion spring or compression spring.

In some example embodiments, alone or in combination with any previous embodiment, a spring is a torsion spring in combination with a compression spring.

In some example embodiments, alone or in combination with any previous embodiment, a torsion spring is a double torsion spring.

In some example embodiments, alone or in combination with any previous embodiment, the incision assembly includes a pivot pin, the pivot pin securing the torsion spring, alone or in combination with the compression spring, and a proximal end of the scalpel within the incision assembly.

In some example embodiments, alone or in combination with any previous embodiment, a double torsion spring straddles the cutting surface.

In some example embodiments, alone or in combination with any previous embodiment, a cutting surface is biased to rotate laterally outward through the at least one opening of the flexible catheter.

In some example embodiments, alone or in combination with any previous embodiment, a cutting surface is biased to pivotably rotate laterally outward from the flexible catheter.

In some example embodiments, alone or in combination with any previous embodiment, a cutting surface has a rounded distal end.

In some example embodiments, alone or in combination with any previous embodiment, a cutting surface has a pointed distal end, the pointed distal end structured to puncture the pericardial tissue from within a pericardial space.

In some example embodiments, alone or in combination with any previous embodiment, a cutting surface is angled between its distal end and its proximal end.

In some example embodiments, alone or in combination with any previous embodiment, an angle is acute.

In some example embodiments, alone or in combination with any previous embodiment, a cutting surface forms an acute angle between its distal end and an outer surface of the flexible catheter.

In some example embodiments, alone or in combination with any previous embodiment, an angle between the cutting surface and the outer surface of the flexible catheter is structured to receive at least a portion of pericardial tissue.

In some example embodiments, alone or in combination with any previous embodiment, a housing includes an anti-buckle mechanism.

In some example embodiments, alone or in combination with any previous embodiment, an anti-buckle mechanism includes a spring axially aligned with the longitudinal axis of the flexible catheter.

In some example embodiments, alone or in combination with any previous embodiment, the incision assembly includes a conductive wire operably coupled to the housing.

In some example embodiments, alone or in combination with any previous embodiment, the conductive wire is operably coupled to the controller.

In some example embodiments, alone or in combination with any previous embodiment, at least a portion of a cutting surface is coupled to the conductive wire.

In some example embodiments, alone or in combination with any previous embodiment, the incision assembly includes at least one stabilizing member.

In some example embodiments, alone or in combination with any previous embodiment, the at least one stabilizing member is operably coupled to the controller.

In some example embodiments, alone or in combination with any previous embodiment, the at least one stabilizing member reversibly projects laterally from the incision assembly.

In some example embodiments, alone or in combination with any previous embodiment, a controller concurrently or sequentially laterally projects the cutting surface and the at least one stabilizing member through the one or more openings of the flexible catheter.

In some example embodiments, alone or in combination with any previous embodiment, at least one stabilizing member reversibly projects laterally about 120 degrees radially apart about the incision assembly.

In some example embodiments, alone or in combination with any previous embodiment, at least one stabilizing member is a wire, loop, or shape-memory metal.

In some example embodiments, alone or in combination with any previous embodiment, at least one stabilizing member is an inflatable structure.

In some example embodiments, alone or in combination with any previous embodiment, the medical device includes at least one nerve detection device.

In some example embodiments, alone or in combination with any previous embodiment, at least one nerve detection device is located on the flexible catheter.

In some example embodiments, alone or in combination with any previous embodiment, at least one nerve detection device is located adjacent the incision assembly.

In some example embodiments, alone or in combination with any previous embodiment, at least one nerve detection device is located on the distal tip.

In some example embodiments, alone or in combination with any previous embodiment, at least one nerve detection device is located on the cutting surface.

In some example embodiments, alone or in combination with any previous embodiment, the medical device includes at least one nerve stimulation device.

In some example embodiments, alone or in combination with any previous embodiment, at least one nerve stimulation device is located on the flexible catheter.

In some example embodiments, alone or in combination with any previous embodiment, at least one nerve stimulation device is located adjacent the incision assembly.

In some example embodiments, alone or in combination with any previous embodiment, at least one nerve stimulation device is located on the distal tip.

In some example embodiments, alone or in combination with any previous embodiment, at least one nerve stimulation device is located on the cutting surface.

In some example embodiments, alone or in combination with any previous embodiment, at least a portion of the distal tip is radiopaque.

In some example embodiments, alone or in combination with any previous embodiment, the cutting surface faces away from the distal end and towards the proximal end of the flexible catheter.

In some example embodiments, alone or in combination with any previous embodiment, an incision assembly is structured to reversibly pivot the cutting surface from a projected configuration to a retracted configuration for providing a scissor-like action on pericardial tissue.

In some example embodiments, alone or in combination with any previous embodiment, the incision assembly has a first configuration with the cutting surface covered by the distal end of the sheath, and a second configuration where the cutting surface is laterally projected when the sheath is longitudinally traversed from the incision assembly.

In some example embodiments, alone or in combination with any previous embodiment, at least a portion of the distal end of the sheath is radiopaque to align the distal end of the sheath and the incision assembly.

In some example embodiments, alone or in combination with any previous embodiment, at least a portion of the cutting surface includes an electrode, and the cutting surface is pivotably projectable so that the electrode is exposed to pericardial tissue.

In some example embodiments, alone or in combination with any previous embodiment, the cutting surface includes an electrode, and the cutting surface is partially pivotably projectable so that at least a portion of the electrode is concealed from pericardial tissue.

In some example embodiments, alone or in combination with any previous embodiment, the electrode is a wire.

In some example embodiments, alone or in combination with any previous embodiment, the wire is shaped as one or more arcs projecting laterally from the flexible catheter along the longitudinal axis.

In some example embodiments, alone or in combination with any previous embodiment, a medical device is provided for creating incisions within tissue, such as a pericardium, the device comprising: a transcatheter structured for location under the tissue, such as in the pericardial cavity; an incision device operably coupled to a distal end of the transcatheter, wherein the incision device is structured for forming an incision in the tissue when brought into contact with the tissue, such as the parietal layer.

### BRIEF DESCRIPTION OF THE DRAWINGS

In order to understand and to see how the present disclosure is carried out in practice, example embodiments will now be described, by way of non-limiting example embodiments only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIGS. 2A-2C depict an exemplary medical device, as disclosed and described herein.

FIGS. 3A and 3B depict the exemplary medical device of FIGS. 2A-2C deployed within a pericardial cavity, as disclosed and described herein.

FIGS. 4A and 4B depict an exemplary incision device deployed within a pericardial cavity, as disclosed and described herein.

FIG. 5 depicts an exemplary incision device deployed within a pericardial cavity, as disclosed and described herein.

FIGS. 6A and 6B depict exemplary control devices for delivering the incision devices disclosed and described herein.

FIG. 7 is a simplified diagram of a transcatheter approach to the pericardial cavity, as disclosed and described herein.

FIG. 8 is a simplified diagram of an alternative transcatheter approach to the pericardial cavity, as disclosed and described herein.

FIG. 9 is a simplified diagram of a parietal layer incision length and cut path as disclosed and described herein.

### DETAILED DESCRIPTION

The present disclosure provides for a catheter-based therapy referred to as transcatheter alleviation of pericardial restraint (TAPR) that can reduce pericardial restraint by incising or opening the pericardium with the intention of improving patient outcomes with heart dysfunction, for example, HFpEF or HFrEF and reducing HF readmissions related thereto. The present disclosure, in one example, provides a device with a concealed/medially-facing cutting surface for accessing and modifying a subject's pericardium for relieving pericardial restraint and/or resolving a heart dysfunction. The present disclosure further provides for methods of treating heart dysfunction using the presently disclosed device.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a transcatheter device or catheter, and the application of a directional force sufficient to cut or separate the tissue, the force being substantially in a direction towards the proximal end of the transcatheter device or catheter, for example, by pulling the transcatheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filleting,” “slicing,” and/or the like.

As used herein the phrase “incision length” is inclusive of a non-zero distance of a cut or incision, for example, beginning at a first point, e.g., a target point, and terminating at a second point, e.g., an access point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “reduced ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “reduced ejection fraction” is inclusive of the ordinary and customary meaning to one of ordinary skill in medical and surgical arts, for example, impairment of ventricular filling or ejection of blood or both, with a clinical syndrome in which patients display left ventricular ejection fraction (LVEF) of 40% or less and are accompanied by progressive left ventricular dilatation and adverse cardiac remodeling and/or mitral valve dysfunction.

As used herein the phrase “heart dysfunction” is inclusive of the ordinary and customary meaning to one of ordinary skill in medical and surgical arts, for example, heart failure or congestive heart failure.

As used herein the phrase “incision device” is inclusive of a device with a cutting surface, for example an edge of a blade or a surface of an electrode.

As used herein the phrase “pericardial incision assembly” and “incision assembly” are used interchangeable and refer to an assemblage that includes an incision device.

As used herein the phrase “transcatheter device” is inclusive of a catheter structured with at least one lumen including a medical instrument, device, or component thereof, for example, an incision device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements is present.

As used herein, when an element is referred to as being “directly coupled” or “directly adjacent” to another element, other elements or intervening elements are not present.

As used herein, term “operably coupled,” includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein the phrase “distal tip” is inclusive of an atraumatic object suitable for puncturing or penetrating tissue without substantial trauma to or bleeding from the vicinity of the picture or penetration.

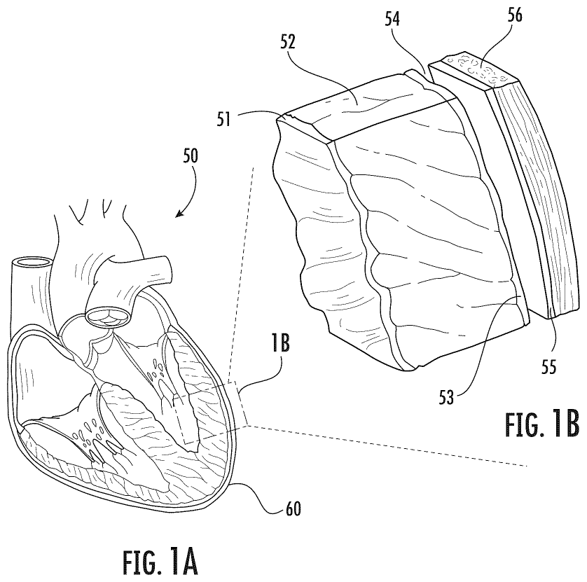

With reference to FIGS. 1A and 1B, section 1B depicts the layers of a heart wall of a heart 50, from inside-out, being the endocardium 51, the myocardium 52, the visceral pericardium 53, the pericardial cavity 54, the parietal pericardium 55, and the fibrous pericardium 56. In some example embodiments, alone or in combination with any previous embodiment, the presently disclosed devices are structured for introduction to the pericardial cavity 54 and for cutting the parietal pericardium 55 and/or the fibrous pericardium 56 and/or the parietal layer.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

With general reference to the figures, a medical device includes a flexible catheter 129 having a distal end, at least one lumen, and a longitudinal axis, an incision device 300, 400, 450, and 500 coupled to the distal end of the catheter 129, and distal tip 115 coupled to and projecting from the incision device 300, 400, 450, and 500. In one example, the distal tip 115 includes a distal tip. In some example embodiments, alone or in combination with any previous embodiment, at least a portion of the flexible catheter 129 tip is radiopaque. Additionally, or alternatively, at least a portion of the incision device 300, 400, 450, and 500 is radiopaque. For example, at least a portion of the distal tip 115 is radiopaque. In some example embodiments, alone or in combination with any previous embodiment, the incision device 300, 400, 450, and 500 includes one or more electrodes structured to receive current and/or radio frequency energy to ablate, burn, vaporize, and/or separate tissue. Additionally, or alternatively, the incision device 300, 400, 450, and 500 includes one or more blades for puncturing and/or cutting tissue. In some example embodiments, alone or in combination with any previous embodiment, the incision device 300, 400, 450, and 500 includes one or more electrodes and one or more blades, and at least one of the electrodes is positioned on and/or adjacent to a cutting surface of at least one of the blades. Additionally, or alternatively, the incision device 300, 400, 450, and 500 includes one or more electrodes and one or more blades, and at least one of the electrodes is electrically isolated from at least one of the blades.

Part or all of the medical device is sterilized for use. The medical device is sterilized using various sterilizing techniques, such as E-Beam sterilization, gamma sterilization, ethylene oxide sterilization, autoclave sterilization, and/or the like. Additionally, one or more materials used in the medical device has anti-bacterial characteristics. The pericardial transection device and/or catheter and/or sheath can be structured such that the total outer diameter (O.D.) introduced to the pericardial cavity is between about 6 Fr (2 mm) and about 30 Fr (10 mm).

In some example embodiments, alone or in combination with any previous embodiment, the medical device is used to cut the parietal pericardium 55 and/or the fibrous pericardium 56 in a series of repeated steps after the medical device is positioned within the pericardial cavity 54. For example, an exemplary method of cutting the parietal pericardium 55 and/or the fibrous pericardium 56 includes steps of positioning an incision device adjacent an initial portion of the parietal pericardium 55 and/or the fibrous pericardium 56, stimulating tissue adjacent the incision device to determine whether the incision device is proximate a portion of the phrenic nerve, measuring an impedance of tissue adjacent the incision device to determine a thickness of the tissue, adjusting, based on the determined thickness, a level of current and/or radio frequency energy to be applied by the incision device to the tissue, applying the level of current and/or radio frequency energy to the incision device to ablate, burn, vaporize, and/or separate tissue, repositioning the incision device adjacent another portion of the parietal pericardium 55 and/or the fibrous pericardium 56, which itself is adjacent the initial portion, and repeating the steps of stimulating, measuring, adjusting, applying the current and/or radio frequency energy, and repositioning. In this way, the medical device is safely and accurately advanced through the parietal pericardium 55, the fibrous pericardium 56, and surrounding tissue without damaging the phrenic nerve, applying excessive current and/or radio frequency energy, and/or damaging other tissue adjacent the parietal pericardium 55 and/or the fibrous pericardium 56 that does not need to be cut.

Additionally, or alternatively, the method includes using the incision device to clamp and/or confine a portion of tissue (e.g., including the parietal pericardium 55 and/or the fibrous pericardium 56) after positioning the incision device and before applying current and/or radio frequency energy to the incision device. In this regard, clamping and/or confining the portion of tissue reduces a surface cross-section of the tissue through which the current and/or radio frequency energy is applied and/or driven, which increases efficiency and/or effectiveness of the cutting and permits focusing of the current and/or radio frequency energy.

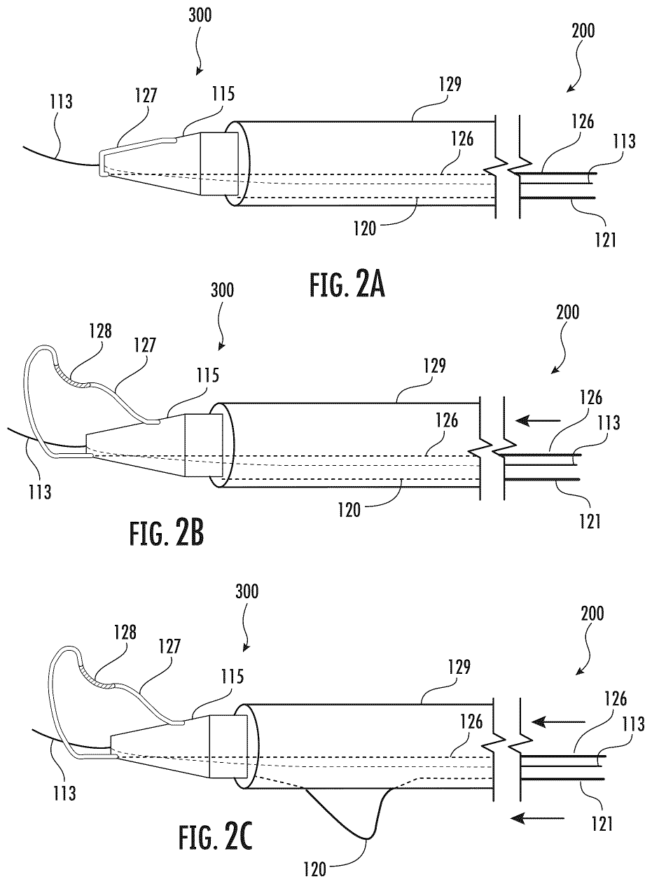

As shown in FIGS. 2A-2C, an exemplary medical device 200 for creating incisions within a pericardium includes a transcatheter 129 (e.g., a flexible catheter), a distal tip 115, and an incision device 300. The transcatheter 129 includes at least one lumen, a longitudinal axis, a proximal end, and a distal end. The distal tip 115 is adjacent the distal end of the transcatheter 129 and includes a body having an external surface and a distal opening. The medical device 200 is deployed within the pericardial cavity 54 in a manner similar to other medical devices described herein.

As shown in FIGS. 2A-2C, the incision device 300 is operably coupled to the distal end of the transcatheter 129. The incision device 300 includes a wire 127 that includes a first end fixed to the external surface of the body of the distal tip 115 and a second end extending through the distal opening of the body of the distal tip 115. The wire 127 includes an electrode 128. In some example embodiments, alone or in combination with any previous embodiment, the electrode 128 is structured to receive current and/or radio frequency energy to ablate, burn, vaporize, and/or separate tissue. Additionally, or alternatively, the electrode 128 has any length (e.g., along the wire 127). For example, the electrode 128 has a length sufficient to correspond to a cross-sectional thickness of the parietal pericardium 55 and/or the fibrous pericardium 56 such that the electrode 128 fully ablates, burn, vaporize, and/or separate tissue in a cross-section of the parietal pericardium 55 and/or the fibrous pericardium 56.

As shown in FIG. 2A, the wire 127 has a first configuration in which a portion of the wire 127 extending from its first end to the distal opening of the body of the distal tip 115 touches (contacts, abuts, is connected to) the external surface of the body. As shown in FIGS. 2B and 2C, the wire 127 has a second configuration in which the portion of the wire 127 extending from the first end to the distal opening of the body of the distal tip 115 forms a shape including a protrusion extending substantially perpendicular to the longitudinal axis of the transcatheter 129. For example, and as shown in FIGS. 2B and 2C, the protrusion extends in an upward direction. As also shown in FIGS. 2B and 2C, when in the second configuration, the electrode 128 of the wire 127 is positioned proximal to the protrusion (e.g., between the protrusion and the transcatheter 129, adjacent a base of the protrusion, and/or the like). While the depicted embodiment illustrates the protrusion extending substantially perpendicular, it is understood that the protrusion can be structured to various different positions at different angles relative to the longitudinal axis of the transcatheter via manipulation of the biasing member (discussed below).

As shown in FIGS. 2A-2C, the medical device 200 includes a biasing member 126, such as an actuating rod, operably coupled to the second end of the wire 127. The biasing member 126 is structured to advance at least a portion of the wire 127 through the distal opening of the body of the distal tip 115 to move the wire 127 from the first configuration to the second configuration. For example, and as shown in FIGS. 2A and 2B, advancing the biasing member in a distal direction parallel to the longitudinal axis advances the wire 127 through the distal opening of the body of the distal tip 115 to move the wire 127 from the first configuration to the second configuration. In some example embodiments, alone or in combination with any previous embodiment, the biasing member 126 is connected to the second end of the wire 127, and, when the biasing member 126 is moved in a distal direction parallel to the longitudinal axis, the biasing member 126 pushes the second end of the wire 127 towards the distal opening of the body of the distal tip 115 such that a greater length of the wire 127 extends beyond the distal opening and permits the wire 127 to take on the shape of the second configuration. Additionally, or alternatively, the biasing member 126 is structured to retract at least a portion of the wire 127 through the distal opening of the body of the distal tip 115 to move the wire 127 from the second configuration to the first configuration.

As shown in FIGS. 2A-2C, the medical device 200 includes a guidewire 113 that feeds through the transcatheter 129 and through the distal opening of the body of the distal tip 115. The guidewire 113 is first be deployed into the pericardial cavity 54, and then the medical device 200 is advanced over the guidewire 113 to a desired position within the pericardial cavity 54 for creating one or more incisions.

As shown in FIGS. 2A-2C, the medical device 200 includes one or more stabilizing members 120, which extends outwardly from the transcatheter 129. For example, and as shown in FIG. 2C, the one or more stabilizing members 120 extends through an opening in the transcatheter 129 after another biasing member 121, such as another actuating rod, a controller rod, and/or an actuating wire, is advanced in a distal direction parallel to the longitudinal axis of the transcatheter 129. The one or more stabilizing members 120 stabilizes the medical device 200 in the pericardial cavity 54 by pressing against the visceral pericardium 53 and/or the parietal pericardium 55, such that the medical device 200 creates an incision in the parietal pericardium 55 and/or the fibrous pericardium 56 in a controlled manner.

In some example embodiments, alone or in combination with any previous embodiment, the one or more stabilizing members 120 is independently user controlled by advancing biasing member 121 distally toward distal tip 115 to laterally extend the one or more stabilizing members 120 a distance from the assembly. In some example embodiments, alone or in combination with any previous embodiment, two or more stabilizing members 120 are positioned radially about the assembly. For example, two or more stabilizing members 120 is positioned radially about the assembly about 120 degrees apart. In some example embodiments, alone or in combination with any previous embodiment, two or more stabilizing members 120 is offset longitudinally from the electrode 128 to minimize or eliminate pushing the incision device 300 through the newly cut slit in the pericardium just as it is formed. The stabilizing members 120 includes flexible rods, flexible strips, and/or inflatable structures, such as balloons that can be inflated with air and/or liquid (e.g., saline).

In some example embodiments, alone or in combination with any previous embodiment, the wire 127 includes a drawn filled tube. For example, the wire 127 includes a core within a shell, and the shell is less conductive than the core. As another example, the wire 127 includes a core within a shell, where the shell is nitinol and the core is silver. As yet another example, the wire 127 includes a core within a shell, and a portion of the shell is removed to expose the core, thereby forming the electrode 128.

In some example embodiments, alone or in combination with any previous embodiment, the wire 127 includes a frame, and the electrode 128 and a conductive wire is attached to the frame. For example, the wire 127 includes a frame having a rectangular cross-section. As another example, the wire 127 includes a nitinol frame.

In some example embodiments, alone or in combination with any previous embodiment, the wire 127 is shape-set to form the second configuration. For example, the wire 127 is shape-set such that the wire 127 takes on the shape of the second configuration when the wire 127 is not under tension but may still be put under tension to become substantially linear. In some example embodiments, alone or in combination with any previous embodiment, the wire 127 includes a shape-memory metal.

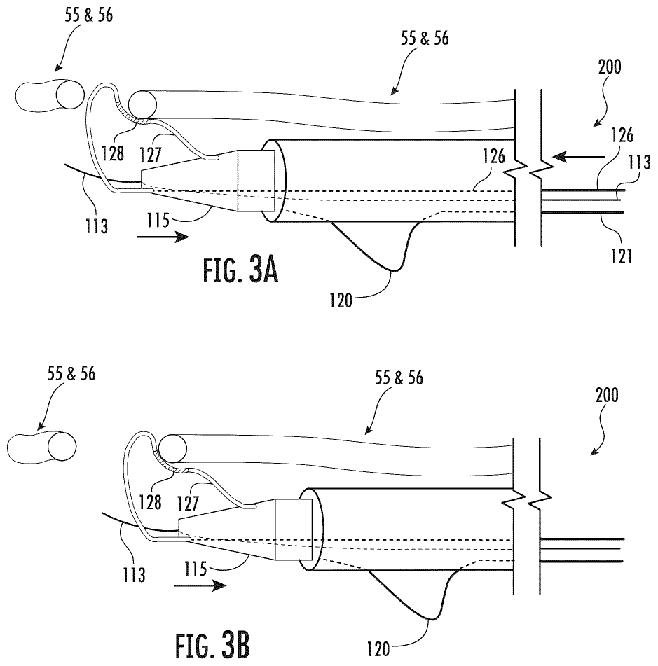

FIGS. 3A and 3B depict the medical device 200 deployed within the pericardial cavity 54 underneath the parietal pericardium 55 and the fibrous pericardium 56. As shown in FIG. 3A, after an opening has been formed in the parietal pericardium 55 and the fibrous pericardium 56 (e.g., by the electrode 128, by a blade of the medical device 200 (not shown), by another medical device (not shown), and/or the like) and the wire 127 has taken on the shape of the second configuration, the protrusion extends through the parietal pericardium 55 and the fibrous pericardium 56. Furthermore, and as also shown in FIG. 3A, when the protrusion extends through the parietal pericardium 55 and the fibrous pericardium 56, the electrode 128 is positioned adjacent to a proximal end of the opening in the parietal pericardium 55 and the fibrous pericardium 56.

In this way, the second configuration in which the protrusion extends perpendicular to the longitudinal axis of the transcatheter 129, permits an operator of the medical device 200 to position the protrusion within the opening and receive tactile and/or visual feedback (e.g., via fluoroscopy and/or the like) indicating positioning of the wire 127 and/or the electrode 128 with respect to the already formed opening in the parietal pericardium 55 and the fibrous pericardium 56. Furthermore, such tactile and/or visual feedback ensures the operator that the electrode 128 is (i) positioned appropriately to continue cutting the parietal pericardium 55 and the fibrous pericardium 56 and (ii) is not positioned such that the electrode 128 is in the opening and/or cuts other tissue upon activation.

As shown in FIG. 3B, the electrode 128 is provided with current and/or radio frequency energy to ablate, burn, vaporize, and/or separate the parietal pericardium 55 and the fibrous pericardium 56 to expand the opening. As also shown in FIG. 3B, the medical device 200 is moved in a proximal direction (e.g., along the guidewire 113) and the protrusion provides tactile and/or visual feedback to the operator regarding the positioning of the wire 127 and/or the electrode 128 with respect to the edge of the opening in the parietal pericardium 55 and the fibrous pericardium 56. In this way, the medical device 200 is moved in either a continuous motion or a stepwise motion in a proximal direction to continue expanding the opening in the parietal pericardium 55 and the fibrous pericardium 56 to expand the opening by activation of the electrode 128, while the protrusion of the wire 127 provides tactile and/or visual feedback to the operator regarding the positioning of the wire 127 and/or the electrode 128.

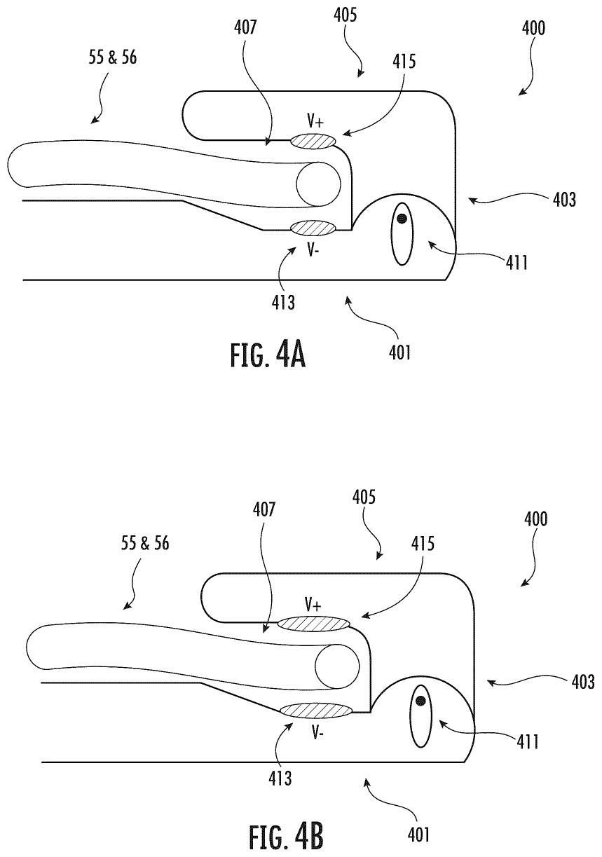

FIGS. 4A and 4B depict exemplary incision devices 400 and 450 for creating incisions within a pericardium. Each of the incision devices 400 and 450 is deployed within the pericardial cavity 54 in a manner similar to other medical devices described herein. Additionally, each of the incision devices 400 and 450 is part of a medical device and is deployed using a flexible catheter.

As shown in FIGS. 4A and 4B, each of the incision devices 400 and 450 includes a base portion 401, a connecting portion 403, and an upper portion 405. In some example embodiments, alone or in combination with any previous embodiment, and as shown in FIGS. 4A and 4B, the base portion 401 includes a distal end and extend in a distal direction substantially parallel to a longitudinal axis. Additionally, or alternatively, and as shown in FIGS. 4A and 4B, the upper portion 405 extends in a proximal direction substantially parallel to the longitudinal axis. As also shown in FIGS. 4A and 4B, the connecting portion 403 joins the upper portion 405 to the distal end of the base portion 401. In other words, the base portion 401 and the upper portion 405 is substantially parallel to each other and be joined together by the connecting portion 403.

As shown in FIGS. 4A and 4B, the base portion 401, the connecting portion 403, and the upper portion 405 forms a confinement space 407 for receiving a portion of the parietal pericardium 55 and the fibrous pericardium 56. In this regard, after an incision is made in the parietal pericardium 55 and the fibrous pericardium 56, the upper portion 405 is extended upward through the incision and positioned above the upper surface of the parietal pericardium 55 and the fibrous pericardium 56, while the base portion 401 remains positioned below the lower surface of the parietal pericardium 55 and the fibrous pericardium 56 (i.e., within the pericardial cavity 54). Alternatively, after an incision is made in the parietal pericardium 55 and the fibrous pericardium 56, the upper portion 405 is extended downward through the incision and positioned below the lower surface of the parietal pericardium 55 and the fibrous pericardium 56 (i.e., within the pericardial cavity 54), while the base portion 401 remains positioned above the upper surface of the parietal pericardium 55 and the fibrous pericardium 56.

As shown in FIGS. 4A and 4B, a portion of the parietal pericardium 55 and the fibrous pericardium 56 is positioned within the confinement space 407. In some example embodiments, alone or in combination with any previous embodiment, and as shown in FIGS. 4A and 4B, each of the incision devices 400 and 450 includes a sliding joint 411 joining the base portion 401 and the upper portion 405 such that a distance between the upper portion 405 and the base portion 401 increases and decreases. Stated differently, the sliding joint 411 permits a height of the confinement space 407 to increase or decrease. For example, such a sliding joint 411 permits each of the incision devices 400 and 450 to accommodate different thicknesses of the parietal pericardium 55 and the fibrous pericardium 56 in the confinement space 407.

As shown in FIGS. 4A and 4B, the incision device 400 includes a first electrode 413 positioned on the base portion 401 and oriented toward the confinement space 407. As also shown in FIG. 4A, the incision device 400 includes a second electrode 415 positioned on the upper portion 405 and oriented toward the confinement space 407 (e.g., opposite the first electrode 413). In some example embodiments, alone or in combination with any previous embodiment, the first electrode 413 and the second electrode 415 has opposite charges. For example, and as shown in FIG. 4A, the first electrode 413 is a negative electrode, and the second electrode 415 is a positive electrode. As will be appreciated by one of ordinary skill in the art, In some example embodiments, alone or in combination with any previous embodiment, the first electrode 413 is a positive electrode, and the second electrode 415 is a negative electrode. Furthermore, the incision device 400 includes only one electrode or more than two electrodes (e.g., three electrodes, four electrodes, five electrodes, six electrodes, and/or the like). As shown by comparing FIGS. 4A and 4B, the first electrode 413 and the second electrode 415 has different lengths in different example embodiments.

In some example embodiments, alone or in combination with any previous embodiment, the first electrode 413 and the second electrode 415 is used to measure an impedance of the tissue positioned within the confinement space 407. For example, after a portion of the parietal pericardium 55 and/or the fibrous pericardium 56 is positioned within the confinement space 407, the first electrode 413 and the second electrode 415 is used as an impedance sensor to determine a thickness and/or composition of the tissue. Additionally, or alternatively, based on the impedance measurement taken by the first electrode 413 and the second electrode 415, a level of current and/or radio frequency energy to be applied by the incision device to the tissue is adjusted.

In some example embodiments, alone or in combination with any previous embodiment, the first electrode 413 and the second electrode 415 is laterally offset, as opposed to vertically offset as shown in FIGS. 4A and 4B. For example, the first electrode 413 and the second electrode 415 is positioned on the connecting portion 403 and be offset from each other in a direction perpendicular to the cross-sectional view shown in FIGS. 4A and 4B. Additionally, or alternatively, the incision device 400 includes another electrode positioned on the upper portion 405 facing in an upward direction in the orientation shown in FIGS. 4A and 4B such that the other electrode is used to create an initial opening in the parietal pericardium 55 and the fibrous pericardium 56 through which the upper portion 405 extends. In some example embodiments, alone or in combination with any previous embodiment, a guidewire is used to puncture the parietal pericardium 55 and the fibrous pericardium 56 to create the initial opening for the upper portion 405.

In some example embodiments, alone or in combination with any previous embodiment, the first electrode 413 and the second electrode 415 is structured to receive current and/or radio frequency energy to ablate, burn, vaporize, and/or separate tissue, such as the parietal pericardium 55 and the fibrous pericardium 56. Because the first electrode 413 and the second electrode 415 are positioned within and oriented toward the confinement space 407, the first electrode 413 and the second electrode 415, when energized, only ablates, burns, vaporizes, and/or separates tissue within the confinement space 407. In other words, by including the confinement space 407 and electrodes oriented toward the confinement space 407, each of the incision devices 400 and 450 protects tissue adjacent to the parietal pericardium 55 and the fibrous pericardium 56 from being damaged.

In some example embodiments, alone or in combination with any previous embodiment, an operator of the incision device 400 or the incision device 450 activates the first electrode 413 and/or the second electrode 415 to ablate, burn, vaporize, and/or separate tissue of the parietal pericardium 55 and the fibrous pericardium 56. Thereafter, the operator advances the incision device 400 or the incision device 450 to position a new portion of the parietal pericardium 55 and the fibrous pericardium 56 in the confinement space 407 (e.g., by pulling the incision device 400 or the incision device 450 to the left in the orientation depicted in FIGS. 4A and 4B). In this regard, the shape of each of the incision devices 400 and 450 provides tactile and/or visual feedback indicating positioning of the parietal pericardium 55 and the fibrous pericardium 56 with respect to the confinement space 407, the first electrode 413, and/or the second electrode 415. For example, if the operator advances the incision device 400 or the incision device 450 too far (e.g., too far to the left in the orientation depicted in FIGS. 4A and 4B), the uncut portion of the parietal pericardium 55 and the fibrous pericardium 56 will abut the connecting portion 403 and prevent further advancement of the incision device 400 or the incision device 450. Such tactile and/or visual feedback ensures the operator that the first electrode 413 and/or the second electrode 415 is (i) positioned appropriately to continue cutting the parietal pericardium 55 and the fibrous pericardium 56 and (ii) is not positioned such that the first electrode 413 and/or the second electrode 415 cuts other tissue upon activation.

In some example embodiments, alone or in combination with any previous embodiment, the first electrode 413 and the second electrode 415 is structured to provide monopolar radio frequency energy to tissue within the confinement space 407. In this regard, current flows from the first electrode 413 and heat tissue adjacent the first electrode 413. Similarly, current flows from the second electrode 415 and heat tissue adjacent the second electrode 415.

Additionally, or alternatively, the first electrode 413 and the second electrode 415 is structured to provide bipolar radio frequency energy to tissue within the confinement space 407. In this regard, current flows from the first electrode 413 to the second electrode 415 and only and/or primarily heat tissue between the first electrode 413 and the second electrode 415. Alternatively, current flows from the second electrode 415 to the first electrode 413 and only and/or primarily heat tissue between the first electrode 413 and the second electrode 415.

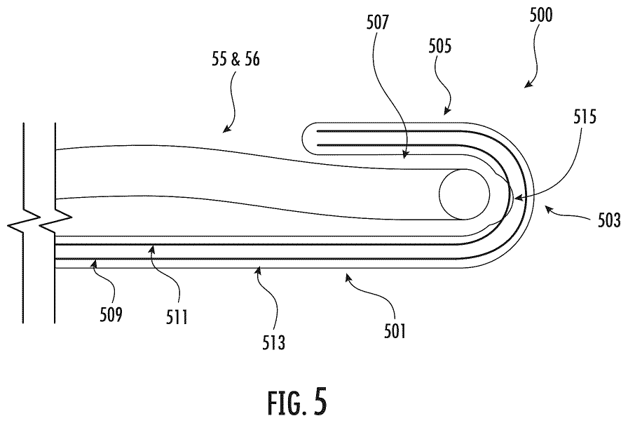

FIG. 5 depicts an exemplary incision device 500 for creating incisions within a pericardium. The incision device 500 is deployed within the pericardial cavity 54 in a manner similar to other medical devices described herein. Additionally, the incision device 500 is part of a medical device and is deployed using a flexible catheter.

As shown in FIG. 5, the incision device 500 includes a base portion 501, a connecting portion 503, and an upper portion 505. In some example embodiments, alone or in combination with any previous embodiment, and as shown in FIG. 5, the base portion 501 includes a distal end and extend in a distal direction substantially parallel to a longitudinal axis. Additionally, or alternatively, and as shown in FIG. 5, the upper portion 505 extends in a proximal direction substantially parallel to the longitudinal axis. As also shown in FIG. 5, the connecting portion 503 joins the upper portion 505 to the distal end of the base portion 501. In other words, the base portion 501 and the upper portion 505 is substantially parallel to each other and be joined together by the connecting portion 503.

As shown in FIG. 5, the base portion 501, the connecting portion 503, and the upper portion 505 forms a confinement space 507 for receiving a portion of the parietal pericardium 55 and the fibrous pericardium 56. In this regard, after an incision is made in the parietal pericardium 55 and the fibrous pericardium 56, the upper portion 505 is extended upward through the incision and positioned above the upper surface of the parietal pericardium 55 and the fibrous pericardium 56, while the base portion 501 remains positioned below the lower surface of the parietal pericardium 55 and the fibrous pericardium 56 (i.e., within the pericardial cavity 54). Alternatively, after an incision is made in the parietal pericardium 55 and the fibrous pericardium 56, the upper portion 505 is extended downward through the incision and positioned below the lower surface of the parietal pericardium 55 and the fibrous pericardium 56 (i.e., within the pericardial cavity 54), while the base portion 501 remains positioned above the upper surface of the parietal pericardium 55 and the fibrous pericardium 56. As shown in FIG. 5, a portion of the parietal pericardium 55 and the fibrous pericardium 56 is positioned within the confinement space 507.

As shown in FIG. 5, the incision device 500 is formed from a first wire 509 and a second wire 511 within an outer sheath 513, and the connecting portion 503 includes a bend in the first wire 509 and the second wire 511. In some example embodiments, alone or in combination with any previous embodiment, the first wire 509 provides rigidity to the incision device 500 and be formed of a material stiffer than the second wire 511. Additionally, or alternatively, the second wire 511 provides an electrical pathway for current and/or radio frequency energy and be formed of a conductive material.

In some example embodiments, alone or in combination with any previous embodiment, the first wire 509 and/or the second wire 511 is flexible and/or shape-set. For example, the first wire 509 and/or the second wire 511 is shape-set such that the first wire 509 and/or the second wire 511 takes on a shape that is curved back on itself as shown in FIG. 5 when the first wire 509 and/or the second wire 511 are not under tension but may still be put under tension to become substantially linear. Such example embodiments may facilitate deployment of the incision device 500 into the pericardial cavity 54 and/or removal of the incision device 500 from the pericardial cavity 54.

In some example embodiments, alone or in combination with any previous embodiment, the outer sheath 513 wraps around and maintain the relative positioning of the first wire 509 and the second wire 511. Furthermore, the outer sheath 513 electrically insulates the majority of the second wire 511. In this regard, a portion of the outer sheath 513 is removed and/or omitted such that an exposed section of the second wire 511 forms an electrode 515 oriented toward the confinement space 507.

Although the incision device 500 shown in FIG. 5 only includes a single electrode 515, the incision device 500, In some example embodiments, alone or in combination with any previous embodiment, includes multiple electrodes (e.g., formed by removing and/or omitting a portion of the outer sheath 513 to expose the second wire 511). Additionally, or alternatively, the incision device 500 also includes one or more electrodes formed using the first wire 509 (e.g., by removing and/or omitting a portion of the outer sheath 513 to expose the first wire 509). In some example embodiments, alone or in combination with any previous embodiment, the first wire 509 and/or the second wire 511 includes a core within a shell, and one or more electrodes is formed by removing a portion of the shell.

In some example embodiments, alone or in combination with any previous embodiment, the electrode 515 is structured to receive current and/or radio frequency energy to ablate, burn, vaporize, and/or separate tissue, such as the parietal pericardium 55 and the fibrous pericardium 56. Because the electrode 515 is positioned within and oriented toward the confinement space 507, the electrode 515, when energized, may only ablate, burn, vaporize, and/or separate tissue within the confinement space 507. In other words, by including the confinement space 507 and one or more electrodes oriented toward the confinement space 507, the incision device 500 may protect tissue adjacent to the parietal pericardium 55 and the fibrous pericardium 56 from being damaged.

In some example embodiments, alone or in combination with any previous embodiment, an operator of the incision device 500 may activate the electrode 515 to ablate, burn, vaporize, and/or separate tissue of the parietal pericardium 55 and the fibrous pericardium 56. Thereafter, the operator may advance the incision device 500 to position a new portion of the parietal pericardium 55 and the fibrous pericardium 56 in the confinement space 507 (e.g., by pulling the incision device 500 to the left in the orientation depicted in FIG. 5). In this regard, the shape of the incision device 500 provides tactile and/or visual feedback indicating positioning of the parietal pericardium 55 and the fibrous pericardium 56 with respect to the confinement space 507 and/or the electrode 515. For example, if the operator advances the incision device 500 too far (e.g., too far to the left in the orientation depicted in FIG. 5), the uncut portion of the parietal pericardium 55 and the fibrous pericardium 56 will abut the connecting portion 503 and prevent further advancement of the incision device 500. Such tactile and/or visual feedback ensures the operator that the electrode 515 is (i) positioned appropriately to continue cutting the parietal pericardium 55 and the fibrous pericardium 56 and (ii) is not positioned such that the electrode 515 may cut other tissue upon activation.

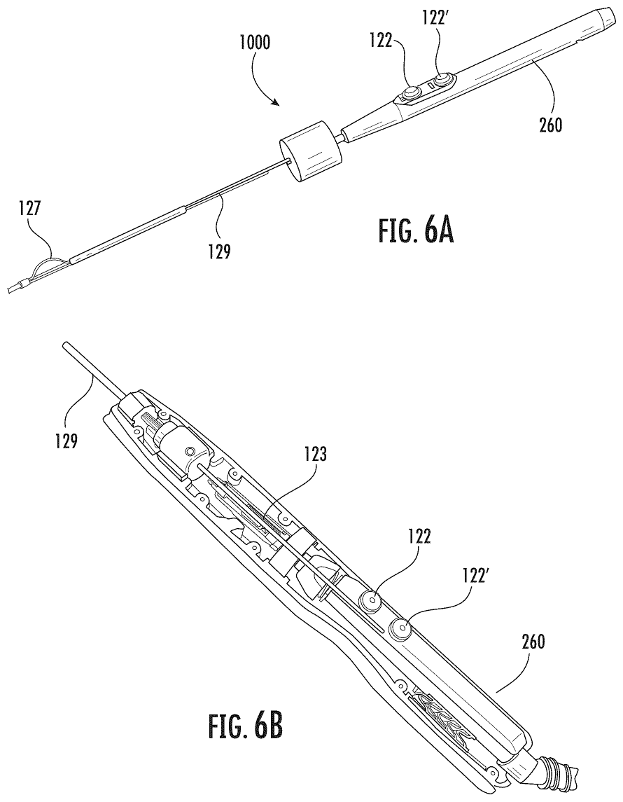

With reference to FIGS. 6A and 6B, a controller 1000 is shown having handle 160, actuating buttons 122, 122′ for operably coupling with the incision device, for example activating one or more electrodes, extending a wire, extending stabilizing members via a biasing member, etc. In one example, the controller 1000 allows the operation of various potential operations of the incision device 300, 400, 450, and 500, including extending and/or retracting a wire, which can be achieved by an appropriate mechanism structured to pull/push a rod. In one example, there is a mechanism used to release/retrieve balloons/nitinol components that function to stabilize and apply counterpressure for the incision device 300, 400, 450, and 500 and its components. Controller 1000 includes one or buttons used to operate and control the electrosurgical features of the device.

Although FIG. 6A depicts wire 127 as a portion of an incision device, one or more of the medical devices and/or incision devices shown and described herein with respect to FIGS. 2A-2C, 3A-3B, 4A-4B, and 5 is coupled to and/or compatible with the controller 1000. Furthermore, although FIGS. 6A and 6B depict a particular controller 1000, the medical devices and/or incision devices shown and described herein with respect to FIGS. 2A-2C, 3A-3B, 4A-4B, and 5 is coupled to and/or compatible with other controllers (e.g., from different manufacturers, vendors, distributors, and/or the like).

As shown in FIG. 6B, the handle 260 may have one or more internal components to transmit the inputs received via the handle (e.g., via engaging actuating buttons 122, 122′) to the catheter (and the incision device on the catheter). For example, the actuating rod 123 may include electrical connectors that connect the actuating buttons 122, 122′ to the catheter (e.g., to provide an electrical current to electrode(s), to move the catheter, to deploy the incision device, etc.). Additionally, the actuating rod 123 may be used to translate the catheter (e.g., in an instance in which the handle 260 includes an actuating knob to control the catheter, the actuating rod 123 may be moved (e.g., rotated and/or moved along the longitudinal axis of the handle 260) to move the catheter).

In one example, at least one incision length is made in the pericardium of a heart. The at least one incision lengths, in a heart with a dysfunction treatable with the present method, may cause the pericardium to separate radially about the cut line, without the need for removal of pericardial tissue. Other incision lengths and paths is employed. Combinations of incision lengths and paths, and combinations of incision lengths and paths with one or more of partial removal of pericardium, drainage, and other pericardial treatments can be employed.

In one example, creating at least one incision length is determined in response to a signal indicative of a reduction of restraint of the heart. In one example, creating at least one incision length is determined in response to a signal indicative of a reduction of restraint of the heart; and repeating the creating of at least one incision length. In one example, the presently disclosed method includes, after creating the at least one incision length, confirming a location of a distal end of the transcatheter device; and in response to a signal indicative of a reduction of restraint of the heart, repeating the steps of creating the at least one incision length, and confirming a location of the distal end.

In one example, a puncture to deliver a guidewire into the pericardial cavity 54 is performed through heart tissue in a transvascular approach. When a transvascular approach through the RAA, IVC, or SVC is employed, a closure device (e.g., occluder) is subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, or SVC using one of the aforementioned transection devices 100, 200, 300, 400, 450, and 500. In one example, a distal tip 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

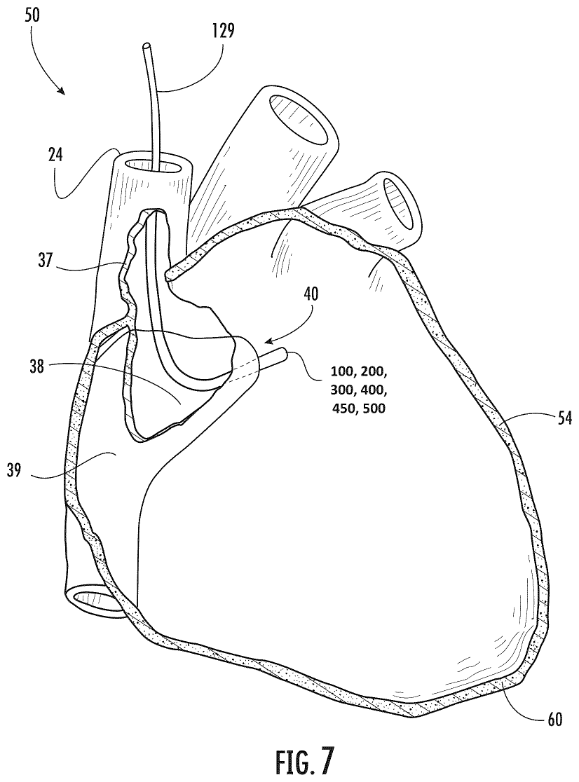

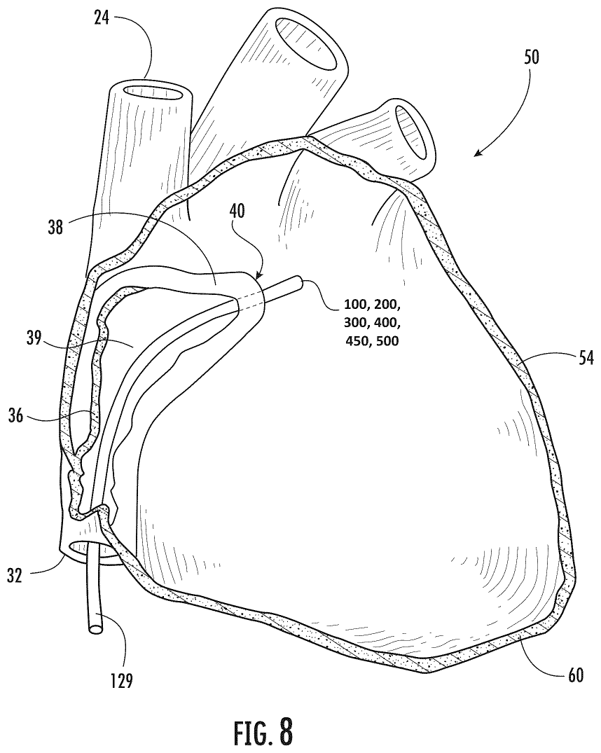

FIGS. 7 and 8 shows exemplary intravascular approaches for delivering the transection devices of the present disclosure to the pericardial cavity 54. Thus, FIG. 7 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by any of the presently disclosed devices substantially without risk of damaging the epicardium or other heart tissue. Other access routes to the pericardial cavity can be used, for example, direct “puncture out” of SVC or IVC/coronary sinus (CS) and a “puncture into” the pericardium.

In some example embodiments, alone or in combination with any previous embodiment, right atrial appendage 38 is accessed via conventional vena cava routes. FIG. 7 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 8 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, a method of reducing pericardial restraint of a subject in need thereof using any of the presently disclosed devices is provided by the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged. Furthermore, left atrial appendage, coronary sinus, and right ventricle pathways are envisaged for transvascular access to the pericardial cavity 54.

Note that the wall of the right atrial appendage is pierced with any of the presently disclosed devices itself, or with an instrument (e.g., guidewire) passed through a lumen of the any of the presently disclosed devices, e.g., over the wire. Further, any of the previously disclosed devices is passed into the pericardial space through the opening in the wall of the atrial appendage, or an instrument passed through the lumen of any of the presently disclosed devices is presented into the pericardial cavity 54. These details will depend on the procedure being performed and on the type of the previously disclosed device being employed.

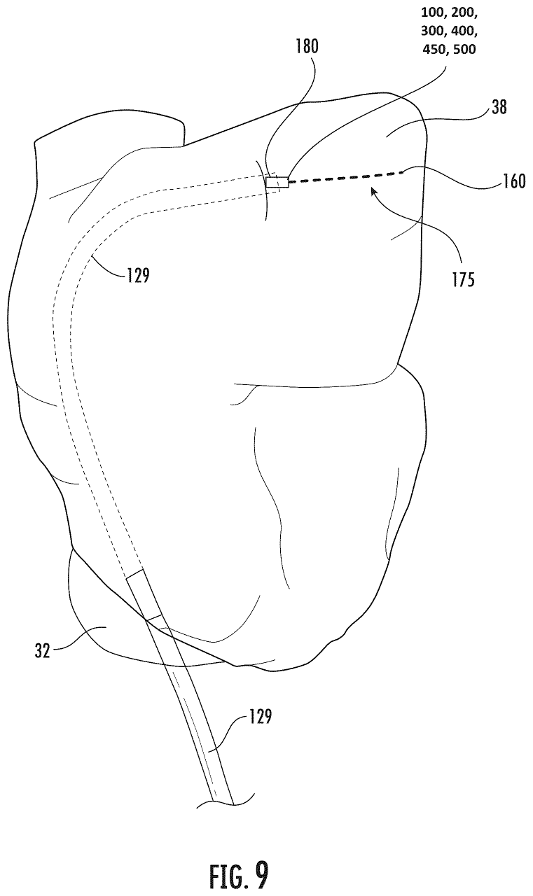

As shown in FIG. 9, any of the presently disclosed devices can be used to create a cut path of a length in a pericardium, e.g., in a parietal layer 58. Thus, a catheter 129, e.g., a steerable catheter can be employed, extending through the IVC, through the RA, and into the RAA, for example, and then into the pericardial cavity 54. The catheter 129 has one or more steerable segments guiding any of the presently disclosed devices, with a radius of curvature of between about 1 inches and about 5 inches, with an arc length of between about 90° and about 180°. As exemplary shown in FIG. 9, any of the presently disclosed devices (e.g., 100, 200, 300, 400, 450, and 500) can be positioned in the pericardial cavity 54 and can begin a cut path 175 at a start point 160 and end at endpoint 180 of a length. At least a portion of the parietal layer 55 of the serous pericardium, and the fibrous pericardium 56, and pericardial adipose tissue 57 are separated along cut path 175. The one or more incisions along the lengths, in a heart with a dysfunction treatable with the present method, cause the pericardium to separate radially about the cut line of the incision path 175, without the removal of pericardial tissue and with a reduction in pericardial restraint. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device includes at least one nerve detection device. In one example, the at least one nerve detection device is located on the flexible catheter 129. In one example, the at least one nerve detection device is located adjacent the incision device. In one example, the at least one nerve detection device is located on the distal tip 115. In one example, the at least one nerve detection device is located adjacent an electrode.

Any one of the presently disclosed devices can further include at least one nerve stimulation device. In one example, the at least one nerve stimulation device is located on the flexible catheter 129. In one example, the at least one nerve stimulation device is located adjacent the incision device. In one example, the at least one nerve stimulation device is located on distal tip 115. In one example, the at least one nerve stimulation device is located adjacent an electrode.

In one example, the presently disclosed devices discussed above includes an optical channel in the transcatheter to accommodate a lens coupled to a fiber optic cable, optionally with a light source, e.g., an LED. In one example, the presently disclosed method further includes obtaining visual information during accessing, traversal of the pericardial cavity, exiting and/or cutting, for example, using an optical channel in the transcatheter to accommodate a lens coupled to a fiber optic cable, optionally with a light source, e.g., an LED.

A kit, including any one of the presently disclosed medical devices, a sheath, a guidewire, and a distal tip is provided.

While certain example embodiments of the present disclosure have been illustrated with reference to specific combinations of elements, various other combinations may also be provided without departing from the teachings of the present disclosure. Thus, the present disclosure should not be construed as being limited to the particular exemplary example embodiments described herein and illustrated in the Figures, but may also encompass combinations of elements of the various illustrated example embodiments and aspects thereof.

We claim:

1. A medical device for creating incisions within tissue, the device comprising:
a transcatheter comprising at least one lumen, a proximal end, and a distal end;
a distal tip adjacent the distal end of the transcatheter, wherein the distal tip comprises a body having an external surface and a distal opening; and
an incision device operably coupled to the distal end of the transcatheter, wherein the incision device comprises:
a wire comprising a first end fixed to the external surface of the body of the distal tip, a second end extending through the distal opening of the body of the distal tip, and an electrode, wherein the wire comprises:
a first configuration in which a portion of the wire extending from the first end of the wire to the distal opening of the body of the distal tip contacts the external surface of the body of the distal tip; and
a second configuration in which (i) the portion of the wire extending from the first end of the wire to the distal opening of the body of the distal tip forms a shape comprising a protrusion extending relative to a longitudinal axis of the transcatheter and (ii) the electrode is positioned proximal to the protrusion.

2. The medical device of claim 1, further comprising a biasing member operably coupled to the second end of the wire, wherein the biasing member is structured to advance at least a portion of the wire through the distal opening of the body of the distal tip to structure the wire in either the first configuration or the second configuration.

3. The medical device of claim 2, wherein the biasing member is structured to retract at least a portion of the wire through the distal opening of the body of the distal tip to move the wire from the second configuration to the first configuration between a range of angles including an angle that is substantially perpendicular to the longitudinal axis of the transcatheter.

4. The medical device of claim 1, wherein the electrode is structured to receive current and/or radio frequency energy to ablate, burn, vaporize, and/or separate tissue.

5. The medical device of claim 1, wherein the incision device is structured such that, after an incision is formed within the tissue, when the wire is in the second configuration, and when the incision device is positioned beneath the tissue, the protrusion extends through the incision within the tissue.

6. The medical device of claim 1, wherein, when the wire is in the second configuration, the electrode is positioned on the wire adjacent a base of the protrusion.

7. The medical device of claim 1, wherein the wire is one of a:
wire comprising a drawn filled tube;
wire comprising a core within a shell, and wherein the shell is less conductive than the core;
wire comprising a core within a shell, and wherein the shell is nitinol and the core is conductive material, such as silver;
wire comprising a core within a shell, and wherein the electrode is formed by removing a portion of the shell;
wire comprising a frame, and wherein the electrode and a wire are attached to the frame;
wire comprising a frame having a rectangular cross-section; or
wire comprising a nitinol frame.

8. The medical device of claim 1, wherein the at least one lumen is structured to receive a guidewire extending through the incision device.

9. The medical device of claim 2, wherein the biasing member exerts a rotary force or torque to a cutting surface of the incision device.

10. The medical device of claim 1, further comprising a sheath, wherein the sheath has a distal end being slidably locatable on the transcatheter and traversable along the transcatheter to align at least one opening in the sheath with the at least one opening of the transcatheter, thereby allowing a cutting surface to laterally project the through both the sheath and the transcatheter.

11. The medical device of claim 1, further comprising one or more stabilizing members located adjacent the at least one opening of the transcatheter.

12. The medical device of claim 11, wherein the one or more stabilizing members reversibly projects laterally about 120 degrees radially apart about the transcatheter.

13. The medical device of claim 10, further comprising one or more stabilizing members located adjacent the at least one opening of the transcatheter, wherein the distal end of the sheath is traversable along the transcatheter to uncover the at least one opening of the transcatheter allowing the one or more stabilizing members to laterally project through the one or more openings of the transcatheter.

14. A medical device for creating incisions within tissue, the device comprising:
a transcatheter comprising at least one lumen, a proximal end, and a distal end;
a distal tip adjacent the distal end of the transcatheter, wherein the distal tip comprises a body having an external surface and a distal opening;
an incision device operably coupled to the distal end of the transcatheter, wherein the incision device comprises:
a wire comprising a first end fixed to the external surface of the body of the distal tip, a second end extending through the distal opening of the body of the distal tip, and an electrode; and
a biasing member operably coupled to the second end of the wire, wherein the biasing member is structured to advance at least a portion of the wire through the distal opening of the body of the distal tip to configure the wire in:
a first configuration in which a portion of the wire extending from the first end of the wire to the distal opening of the body of the distal tip contacts the external surface of the body of the distal tip; and
a second configuration in which (i) the portion of the wire extending from the first end of the wire to the distal opening of the body of the distal tip forms a shape comprising a protrusion extending relative to a longitudinal axis of the transcatheter and (ii) the electrode is positioned proximal to the protrusion.

15. The medical device of claim 14, wherein the biasing member is structured to retract at least a portion of the wire through the distal opening of the body of the distal tip to move the wire from the second configuration to the first configuration between a range of angles including an angle that is substantially perpendicular to the longitudinal axis of the transcatheter.

16. The medical device of claim 14, wherein the electrode is structured to receive current and/or radio frequency energy to ablate, burn, vaporize, and/or separate tissue.

17. The medical device of claim 14, wherein the wire comprises:
a base portion comprising a distal end, wherein the base portion extends in a distal direction substantially parallel to the longitudinal axis;
an upper portion extending in a proximal direction substantially parallel to the longitudinal axis; and
a connecting portion joining the upper portion to the distal end of the base portion such that the base portion, the upper portion, and the connecting portion form a confinement space, and
wherein the electrode is oriented toward the confinement space.

18. A medical device for creating incisions within tissue, the device comprising:
a transcatheter comprising at least one lumen, a proximal end, and a distal end;
a distal tip adjacent the distal end of the transcatheter, wherein the distal tip comprises a body having an external surface and a distal opening; and
an incision device operably coupled to the distal end of the transcatheter, wherein the incision device comprises:
a wire comprising a first end fixed to the external surface of the body of the distal tip, a second end extending through the distal opening of the body of the distal tip, and an electrode, wherein the wire comprises:
a base portion comprising a distal end, wherein the base portion extends in a distal direction substantially parallel to the longitudinal axis;
an upper portion extending in a proximal direction substantially parallel to the longitudinal axis; and
a connecting portion joining the upper portion to the distal end of the base portion such that the base portion, the upper portion, and the connecting portion form a confinement space, and
wherein the electrode is oriented toward the confinement space.

19. The medical device of claim 18, wherein the wire is configurable in:
a first configuration in which a portion of the wire extending from the first end of the wire to the distal opening of the body of the distal tip contacts the external surface of the body of the distal tip; and
a second configuration in which (i) the portion of the wire extending from the first end of the wire to the distal opening of the body of the distal tip forms a shape comprising a protrusion extending relative to a longitudinal axis of the transcatheter and (ii) the electrode is positioned proximal to the protrusion.

20. The medical device of claim 19, further comprising a biasing member operably coupled to the second end of the wire, wherein the biasing member is structured to advance at least a portion of the wire through the distal opening of the body of the distal tip to structure the wire in either the first configuration or the second configuration.
